(19) United States
(12) Patent Application Publication
(10) Pub. No.: US 2010/0036233 A1
(43) Pub. Date: Feb. 11, 2010

(54) Automatic Methods for Combining Human Facial Information with 3D Magnetic Resonance Brain Images

(75) Inventors: David Zhu, East Lansing, MI (US); Dirk Colbry, Tempe, AZ (US)

Correspondence Address: Peter G. Carroll;MEDLEN & CARROLL, LLP, Suite 350, 101 Howard Street, San Francisco, CA, 94105, US

(73) Assignee: Michigan State University

(21) Appl. No.: 12/188,352

(22) Filed: Aug. 8, 2008

(51) Int. Cl. A61B 5/055; G06F 7/14; G06F 17/30; G09G 5/00; G06K 9/00

(52) U.S. Cl. 600/410; 707/101; 345/629; 382/128

(57) ABSTRACT

The present invention relates to compositions and methods for provided fused images of external and internal body scans by fusing data generated from color external laser scans with internal magnetic resonance (MR) images. In particular, the present inventions relate to software for fusing and displaying images produced by fusing color 2.5D laser scans of the human face with 3D magnetic resonance (MR) brain images. Further, these fused images may be used for clinical and research applications, such as in methods for neurosurgical planning procedures.

### FIELD OF THE INVENTION

The present invention relates to compositions and methods for providing fused images of external and internal body scans by fusing data generated from color external laser scans with internal magnetic resonance (MR) images. In particular, the present inventions relate to software for fusing and displaying images produced by fusing color 2.5D laser scans of the human face with 3D magnetic resonance (MR) brain images. Further, these fused images may be used for clinical and research applications, such as in methods for neurosurgical planning procedures.

### BACKGROUND OF THE INVENTION

Biometric face recognition technologies are new and evolving systems that governments, airports, firms, and schools are using to identify criminals and protect innocent people. This led to a relatively new process of facial imaging that was developed in which a 3D camera is used to take a series of rapid laser images of individual faces. The laser is completely safe and will not cause any harm to any part of the body including the eyes. Specifically, a subject is seated in a chair and asked to remain still while a camera takes rapid images. The chair is rotated slightly for a 360-degree picture. The data is then collected and “sewn” together in a computer and a file is created to form the image in the desired size and format. However, these captured images are restricted to external body features.

In contrast, magnetic resonance imaging (MRI) scans are used as powerful diagnostic and research tools for capturing internal images. Specifically, MRI is a radiology technique that uses magnetism, radio waves, and a computer to produce images of body structures. An MRI device is a tube surrounded by a giant circular magnet where a patient is placed on a moveable bed that is inserted into the magnet. The magnet creates a strong magnetic field that aligns the protons of hydrogen atoms, which are then exposed to a beam of radio waves. This spins the various protons of the body which produces a faint signal that is detected by the receiver portion of the MRI device. The receiver information is processed by a computer, and an image is produced and typically displayed on a computer screen, either in real-time or statically, with static images recorded on film for diagnostic or research use. The image and resolution produced by MRI is quite detailed and can detect tiny changes of structures within the body. For some procedures, contrast agents, such as gadolinium, are used to increase the accuracy of the images.

An MRI scan is used as an extremely accurate method of disease detection throughout the body. In the head, trauma to the brain can be seen as bleeding or swelling. Other abnormalities often found include brain aneurysms, stroke, tumors of the brain, as well as tumors or inflammation of the spine. Neurosurgeons use an MRI scan in defining brain anatomy. Often, surgery can be deferred or more accurately directed after knowing the results of an MRI scan.

Currently, side-by-side comparisons may be made with external and internal images. However it is difficult and time-consuming and results in imprecise images.

### SUMMARY OF THE INVENTION

The present invention relates to compositions and methods for provided fused images of external and internal body scans by fusing data generated from color external laser scans with internal magnetic resonance (MR) images. In particular, the present inventions relate to software for fusing and displaying images produced by fusing color 2.5D laser scans of the human face with 3D magnetic resonance (MR) brain images. Further, these fused images may be used for clinical and research applications, such as in methods for neurosurgical planning procedures.

The present invention is not limited to any particular system, comprising software, wherein said software is configured to: i) receive a first data set obtained from a digital laser scanner; ii) receive a second data set obtained from a magnetic resonance imaging (MRI) device; and iii) fuse said first data set and said second data set into a fused data set.

In one embodiment, the invention provides software further configured to display said fused data set. In one embodiment, said first data set comprises a human head and neck scan. In one embodiment, said first data set further comprises a human face scan. In one embodiment, said second data set comprises at least one brain anatomy image. In one embodiment, said second data set comprises at least one brain activation image. In one embodiment, said fused data comprises said face and head scan superimposed with said brain anatomy image. In one embodiment, said fused data comprises said face and head scan superimposed with said brain activation image. In one embodiment, said digital laser scanner device is a facial scanner device. In one embodiment, said second data set obtained from a MRI imaging device is in real-time. In one embodiment, said first data set and said second data set sample are obtained from the same subject. In one embodiment, said anatomy image comprises an abnormal cell image. In one embodiment, said activation image comprises an abnormal activation image.

In one embodiment, the invention provides a system, comprising: a) a digital laser scanner device, b) a magnetic resonance imaging (MRI) device, c) a first data set and a second data set, wherein said first data set comprises sample data obtained by a digital laser scanner device and said second data set comprises sample data obtained by a MRI device, d) software, wherein said software is configured to: i) receive a first data set obtained from a digital laser scanner; ii) receive a second data set obtained from a magnetic resonance imaging (MRI) device; and iii) fuse said first data set and said second data set into a fused data set. In one embodiment, said software is further configured to display said fused data set.

In one embodiment, the invention provides a method of generating fused sample data, comprising, a) providing, i) a first data set obtained from a digital laser scanner device, ii) a second data set obtained from a magnetic resonance imaging (MRI) device, and b) combining said first data set and said second data set so as to generate a fused data set. In one embodiment, the invention provides a fused sample data set generated according to the methods provided herein.

In one embodiment, the invention provides a method of generating a display of a fused sample data, comprising, a) providing, i) a first sample data set obtained from a digital laser scanner device, ii) a second sample data set obtained from a magnetic resonance imaging (MRI) device, and iii) a software package configured to fuse sample data sets, and b) combining said first sample data set and said second sample data set using said software package for providing a fused sample data set. In a further embodiment, said method further comprises, iv) a software package capable of displaying a fused data set and c) displaying said fused sample data set. In one embodiment, said first sample data set comprises scanner device data obtained from scanning a human head and neck.

In one embodiment, the invention provides a method of fusing sample data, comprising, a) providing, i) a first data set obtained from a digital laser scanner device, ii) a second data set obtained from a magnetic resonance imaging (MRI) device, and iii) a software package configured to fuse sample data sets, and b) combining said first sample data set and said second sample data set using said software package for providing a fused sample data set. In one embodiment, said method further comprises, iv) a software package capable of displaying a fused data set and c) displaying said fused sample data set. In one embodiment, said first data set comprises a human head and neck scan. In one embodiment, said first data set further comprises a human face scan. In one embodiment, said second data set comprises at least one brain anatomy image. In one embodiment, said second data set comprises at least one brain activation image. In one embodiment, said anatomy image comprises an abnormal cell image. In one embodiment, said activation image comprises an abnormal activation image. In one embodiment, said fused data comprises said face and head scan superimposed with said brain anatomy image. In one embodiment, said fused data comprises said face and head scan superimposed with said brain activation image. In one embodiment, said digital laser scanner device is a facial scanner device. In one embodiment, said sample data set obtained from a MRI imaging device is real-time data. In one embodiment, said first data set and said second data set sample are obtained from the same subject. In one embodiment, said method further comprises, a third sample data set obtained from an event-related potential (ERP) electrode cap and combining said third sample data set with said software for providing a fused sample data set.

In one embodiment, the invention provides a method, comprising, a) providing, i) a human patient, wherein said human patient is in need of a neurosurgical procedure, ii) a surgical incision marker, wherein said marker is capable of showing the location of a planned incision site and the surgical direction of a planned incision site, wherein said marker is capable of being digitally scanned on a human patient, iii) a 3D data set, wherein said 3D data set was obtained by scanning a human patient, iv) a digital laser scanner, capable of generating a 2.5D data set from a human patient, and v) a software package configured to fuse and display a 2.5D data set fused with a 3D data set, wherein said software is further configured to simulate a surgical path on said display, b) marking the location of a planned incision site and the surgical direction of incision with said marker on said human subject, c) scanning said marked patient with said scanner for obtaining a 2.5D data set, d) fusing said 2.5D data set with said 3D data set with said software package, e) displaying said fused data sets, and f) simulating a surgical path on said display for a neurosurgical procedure. In one embodiment, said 3D data set was obtained by a magnetic resonance imaging (MRI) device for capturing said 3D data set from said human subject. In one embodiment, said method further comprises, erasing said marking and repeating steps b)-f), wherein said planned incision site is changed. It is not meant to limit said neurosurgical procedure. Indeed, said neurosurgical procedure includes but is not limited to a brain tumor resection, a craniotomy, a craniosynostectomy, a deep brain stimulator, and the like.

### Definitions

To facilitate an understanding of the present invention, a number of terms and phrases are defined below:

As used herein, the singular forms “a,” “an” and “the” include plural references unless the content clearly dictates otherwise.

As used herein, the terms “processor,” “imaging software,” “software package,” or other similar terms are used in their broadest sense. In one sense, the terms “processor,” “imaging software,” “software package,” or other similar terms refer to a device and/or system capable of obtaining, processing, and/or viewing, and/or superimposing images obtained with an imaging device. As such, software comprises an “algorithm” used in its broadest sense to refer to a computable set of steps to achieve a desired result.

As used herein, the term “configured” refers to a built in capability of software to achieve a defined goal, such as software designed to fuse data sets of the present inventions, to provide fused images of the present inventions, to provide images superimposed with maps of the present inventions, and the like.

As used herein, the term “computer system” refers to a system comprising a computer processor, computer memory, and a computer video screen in operable combination. Computer systems may also include computer software.

As used herein, the term “display” or “display system” or “display component” refers to a screen (e.g., monitor) for the visual display of computer or electronically generated images. Images are generally displayed as a plurality of pixels. In some embodiments, display systems and display components comprise “computer processors,” “computer memory,” “software,” and “display screens.”

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “magnetic resonance imaging (MRI) device” or “MRI” incorporates all devices capable of magnetic resonance imaging or equivalents. The methods of the invention can be practiced using any such device, or variation of a magnetic resonance imaging (MRI) device or equivalent, or in conjunction with any known MRI methodology.

As used herein, the term “scan” refers to a process of traversing a surface with a beam of light, laser, electrons, and the like, in order to provide, reproduce or transmit an image of the surface, for example, a probe scan, a target scan, a head and neck scan, a facial scan, et cetera of the present inventions. Scan may also refer to the resulting data set obtained from the surface.

As used herein, the term “brain anatomy” refers to a location of structures of the brain, such as Basal Ganglia, Brainstem, Broca's Area, Central Sulcus (Fissure of Rolando), Cerebellum, Cerebral Cortex, Cerebral Cortex Lobes, Frontal Lobes, Insula, Occipital Lobes, Parietal Lobes, Temporal Lobes, Cerebrum, Corpus Callosum, Cranial Nerves, Fissure of Sylvius (Lateral Sulcus), Inferior Frontal Gyrus, Limbic System, Amygdala, Cingulate Gyrus, Fornix, Hippocampus, Hypothalamus, Olfactory Cortex, Thalamus, Medulla Oblongata, Meninges, Olfactory Bulb, Pineal Gland, Pituitary Gland, Pons, Reticular Formation, Substantia Nigra, Tectum, Tegmentum, Ventricular System, Aqueduct of Sylvius, Choroid Plexus, Fourth Ventricle, Lateral Ventricle, Third Ventricle, Wemicke's Area, et cetera. “Brain anatomy image” refers to a visualization of brain structures.

As used herein, the term “brain activation” refers to areas of brain activity, whereas a brain activation image or map refers to a visualization of brain activity, such as described and used herein.

As used herein, the term “subject” refers to any animal (e.g., a mammal), including, but not limited to, humans, non-human primates, rodents, and the like, which is to be the recipient of a particular treatment, such as a scan of the present inventions. Typically, the terms “subject” and “patient” are used interchangeably herein in reference to a human or human subject.

As used herein, the term “abnormal” in reference to a cell or activation or function refers to a cell or groups of cells, such as a tissue, that is different than a cell or group of cells, or a tissue than typically observed in an equivalent area of human subject. For example, an abnormal cell may be a cell that is larger or smaller, or larger in number or absent, or that is a cancer cell. A specific example is a brain cell that is dying, or a brain cell or tissue that is either more active or is overactive compared to an equivalent brain cell or tissue.

As used herein, the term “2D” or “two Dimensional” in reference to a scan, refers to a digital shape of physical object as an image captured from a device, comprising coordinates of X (width) & Y (height).

As used herein, the term “2.5D” in reference to a scan refers to a scan comprising both Cartesian (x, y, z) coordinates and color (red, green, blue) information for each recorded pixel within the scan data.

As used herein, the term “3D” or “3 dimensional” in reference to a scan, refers to a digital shape of physical object as an image captured from a device, such as an MRI device, a 3D digitizer, such as a laser scanner, and the like, comprising coordinates of X (width), Y (height), and Z (depth).

As used herein, the term “XYZ Coordinates” refers to a set of numbers that locate a point in a three-dimensional Cartesian coordinate system. For one example, XYZ coordinates may define the set of approximately 300,000 data points from a 3D body scan.

As used herein, the term “data set” refers to a plurality of numbers generated by a digital device, such as facial scanner, MRI device and the like.

As used herein, the term “image” in reference to a computer term refers to a displayable file.

As used herein, the term “map” in reference to a computer term, refers to a file or screen image whose regions comprise specific coordinates for the given image for example, a computer screen image comprising a region that links to a specific URL, an image that maps a region to a specific area of the brain.

As used herein, the term “fuse” or “fusing” or “fusion” refers to superimposing at least 2 images upon one another for correlating the location of specific image features relative to one another rather than side by side comparison of superimposed images. As used herein, the term “superposition” in reference to images, such as “superimposition of images” of specifically related subject matter involves registration of the images and fusion of the images.

As used herein, the term “registration” refers generally to a spatial alignment of at least 2 images after which fusion is performed to produce the integrated display of the combined images. The combined or fused images might be, stored, displayed on a computer screen or viewed on some form of hard output, such as paper, x-ray film, or other similar mediums. Some examples of registration methods includes identification of salient points or landmarks, such as geometric facial points (for example, two scans using automatically detected anchor points, such as the tip of the nose and the inside corners of the eyes as described herein), alignment of segmented binary structures such as object surfaces (for example, markers) and utilizing measures computed from the image grey values, for example, voxel based. Another registration method involves the use of markers, such as fiducials, or stereotactic frames. When using extrinsic methods of image capturing, markers or reference frames are placed next to or onto a patient during imaging. The patient is imaged in each modality where the markers or frames are visible in the image.

As used herein, the term “surgical path” refers to projected or actual incision made by a surgeon, in particular, a neurosurgeon.

As used herein, the term “marker” in reference to a surgical incision marker, refers to a one or more of a probe, ink marker, felt tip marker, and the like.

The term “erasing” in reference to a marker, refers to the removal of the marker, for example by using an alcohol solution to remove a felt tip marker.

As used herein, the term “voxel” refers to a volume pixel, for example, the smallest distinguishable box-shaped part of a three-dimensional image.

### DESCRIPTION OF THE DRAWINGS

FIG. 1 shows an exemplary 2D color facial image produced by a Minolta Vivid 910 scanner where (a) the image comprises x, y and z point values for every visible surface point on the 2D color image, where x, y and z values were stored as matrixes (shown by exemplary images (b), (c) and (d) correspondingly) with rows and columns corresponding to the 2D image.

FIG. 2 shows an exemplary outline of a two-step alignment process used by the 3DID identification system.

FIG. 3 shows exemplary 3D Magnetic Resonance (MR) images and activation maps acquired from a functional MRI (fMRI) experiment: where (a) shows a MRI brain images in axial and coronal views are shown with brain activation maps superimposed (overlaid). Regions preferentially more active to Full Scenes over Faces are shown in red and yellow, and regions preferentially more active to Faces over Full Scenes are shown in blue. (b) The surface of the human head can be viewed by necessary rendering and window viewing level.

FIG. 4 shows exemplary images of an artificial face scan where voxels at the skin regions on the 3D MR images (red dots in left image) were used to create an artificial 2.5D face scan (center). The right image shows a 3D rendering of this depth map.

FIG. 5 shows an exemplary testing database of 36 2.5D scans taken over a four-year period. The average alignment error (mm) for each scan is shown below the corresponding picture. A larger alignment error occurs when the subject is far away from the scanner, changes pose, or changes expression. This database was applied to illustrate the robust nature of the 3DID face alignment algorithm in fusing human face taken from a digital camera to 3D MR images.

FIG. 6 shows exemplary 2.5D images (left two images) acquired from a laser scanner were successfully fused to the high-resolution MR images. The event related potential (ERP) electrode cap worn by the subject did not appear to affect the accurate fusion of data sets. The fused 3D images were overlaid with the brain activation maps (right two images) shown in FIG. 3.

FIG. 7 shows an exemplary schematic of data fusion of a surface map generated from a digital laser scanner in relation to 3D MR brain images.

FIG. 8 shows an exemplary schematic of a flow chart of a direct visual feedback system for neurosurgical planning.

### DETAILED DESCRIPTION OF THE INVENTION

The present invention relates to compositions and methods for provided fused images of external and internal body scans by fusing data generated from color external laser scans with internal magnetic resonance (MR) images. In particular, the present inventions relate to software for fusing and displaying images produced by fusing color 2.5D laser scans of the human face with 3D magnetic resonance (MR) brain images. Further, these fused images may be used for clinical and research applications, such as in methods for neurosurgical planning procedures.

The inventors established the concept and implementation of using facial geometric (external) points, such as from a data set captured by a digital laser scanner for creating a 2.5D face surface scan, as the link to combine data from multiple sources (including external and internal) with high-resolution three-dimension (3D) images, specifically including but not limited to magnetic resonance (MR) images. Further, these fusion methods are contemplated for application to any data set with known geometric relationships to the surface of the human face obtained from imaging devices, for example, functional imaging modalities include scintigraphy, functional MRI (fMRI) and nuclear medicine imaging techniques such as single photon emission computed tomography (SPECT), positron emission tomography (PET), perfusion MRI (pMRI), functional CT (fCT), electro impedance tomography (EIT), magnetic resonance elastography (MRE), electroencephalogram (EEG), and the like.

There are many limitations on methods used to compare both external and internal data, such as MRI images with external markers and ERP information, for diagnostic or other uses. For example, MR brain images were acquired with specially designed and attached external devices. These devices provided partial MR signal for localization purposes. Further, data from different sources are usually directly compared in an attempt to register the information together, such as when data from fMRI and ERP are compared side-by-side but not registered together or combined for display. However one example for combining a display of internal organs is described in U.S. Pat. No. 7,117,026, herein incorporated by reference.

In a further neurosurgery related example, high-resolution MRI scans are taken, and then the neurosurgeon marks the estimated locations where the surgery probes should be applied on the skull surface. In some cases, neurosurgeons take MRI scans with markers attached to the patient's head. These markers provide partial MRI signal for localization. These methods are imprecise yet over 200,000 brain surgical procedures were performed in the United States in 1999, based on the statistics provided by Neurosurgery Today (AANS National Neurosurgical Statistics Report—1999 Procedural Statistics. Posted at a website on the world wide web: www.neurosurgerytoday.org/what/stats/procedures.asp; herein incorporated by reference). Most important to the success of such procedures is careful planning to minimize invasion of normal brain regions.

High resolution 3D MR images are often used as crucial components of neurosurgical planning procedures (Burtscher et al., Comput Aided Surg. 1998;3:27-32; herein incorporated by reference). They allow for 3D visualization of both brain anatomy and the abnormal region targeted for surgery. Additionally, brain functional MR imaging (fMRI) is helpful (Lee et al., AJNR Am J Neuroradiol. 1999;20:1511-1519; herein incorporated by reference), by providing brain activation maps surgeons can minimize invasion to healthy brain regions that have a high impact on the quality of life, such as neuronal tissue and cells for primary sensory, motor and language comprehension and expression (Hirsch et al., Neurosurgery. 2000;47:711-721; discussion 721-722; herein incorporated by reference). Currently, practitioners use imaging information to mark the incision sites with ink on the head surface, or plan the incision site and direction using an external mechanic device attached to the head (Sweeney et al., et al., Strahlenther Onkol. 2003; 179:254-260; herein incorporated by reference).

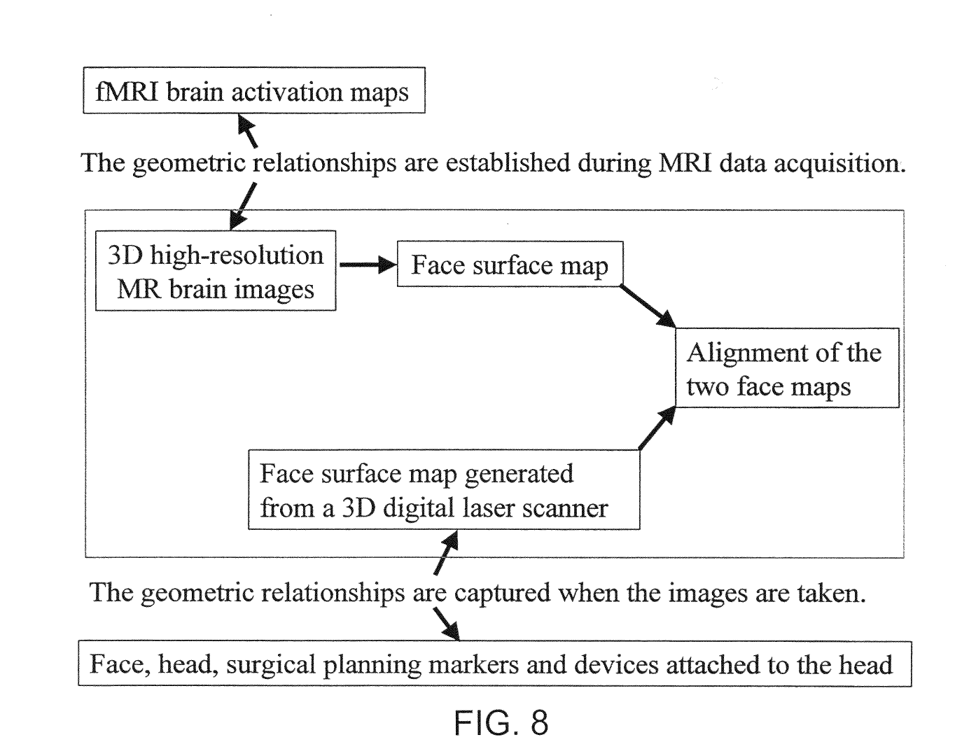

These techniques are helpful, but they do not allow neurosurgeons to directly visualize the incision site and surgical direction with respect to brain anatomy. Because there is no direct visual feedback to help neurosurgeons perfect their accuracy prior to making an incision, there is high reliance on each neurosurgeon's personal ability to mentally visualize the patient's brain anatomy. The real-time direct visual feedback system, and method thereof, described herein (for example, see FIG. 8), is contemplated to provide a significant improvement in the planning of neurosurgical procedures by providing visualization support to neurosurgeons, reducing errors costly in human and monetary measures, and most importantly, improving patient outcome following surgery. In particular, the inventors developed a method perfecting a neurosurgeon's surgical path accuracy prior to making an incision. Prior to neurosurgery a patient is scanned by an MRI device for capturing a 3D internal scan. An incision site is then drawn (marked) on the patient for scanning into a 2.5D data set for fusing with the 3D scan. This fused data set is displayed as a surgical simulation for allowing a neurosurgeon to perfect surgical accuracy prior to making an incision.

In summary, the inventors developed herein a prototype that allows visualized features of the face and head (and any attached devices or frames) superimposed with the brain anatomy and brain activation maps. The prototype software provided a fast, robust and fully automated technique that fused color 2.5D scans of the human face (generated by a 3D digital camera) with a 3D MR brain images. This fusion was highly accurate with alignment errors estimated at 1.4±0.4 mm.

These methods are further contemplated for stereotactic (or similar) neurosurgery planning. A contemplated embodiment includes the fusion of data obtained from evoke-related potential (ERP) experiments with the data obtained from functional MR imaging (fMRI) experiments, for providing benefits of combining high-temporal resolution of the EPR data with high-spatial resolution of fMRI data.

Software comprising an algorithm for aligning human faces in three dimensions was developed by one of the inventors and implemented within a face-verification system called 3DID. The 3DID system matches pairs of 2.5D face scans that was originally developed for biometric research [Colbry et al., The 3DID face alignment system for verifying identity, Image and Vision Computing; and Colbry and Stockman, Identity verification via the 3DID face alignment system. In Proceedings of the IEEE Workshop on Applications of Computer Vision (WACV), Austin, Tex., February 2007; herein incorporated by reference], and security applications [Colbry and Stockman, “Identity Verification via the 3DID Face Alignment System,” Proceedings of the IEEE Workshop on Applications of Computer Vision (WACV), 2007,” wacv, p. 2, Eighth IEEE Workshop on Applications of Computer Vision (WACV'07); herein incorporated by reference]. 3DID was tested on the Face Recognition Grand Challenge [Phillips, et al., “Overview of the Face Recognition Grand Challenge,” in Proceedings IEEE Conference on Computer Vision and Pattern Recognition, 2005; herein incorporated by reference] database of 948 face scans from 275 people, which showed an accuracy of 98.8% for face recognition, with a reject rate of 1.5%.

While the 3DID system was developed using face images generated by a laser scanner, accurate 2.5D face scans were also reconstructed from high-resolution volumetric magnetic resonance (MR) images of the brain. In one embodiment, software comprising an alignment algorithm was used in 3DID and then applied by the inventors herein, to fuse a face surface map from magnetic resonance imaging (MRI) with that obtained by a digital laser scanner. In order to provide a fused image, data from at least two modalities were registered together, such that a functional MRI (fMRI) brain activation data map was overlaid onto facial data. The inventors further provided a true and accurate direct visualization (registry) of the geometric relationships of the head surface directly with face surface data captured from at least two different sources. The inventors provide herein compositions and methods providing a more accurate, more convenient and automated procedure for comparing external and internal images, in addition to combing images obtained from at least two imaging devices, for example, the fusion of at least 2 images, one facial (external) image captured with a digital facial scanner with an MRI (internal) image, was achieved within seconds. Further, the fast fusion process, in seconds, would provide real time methods for visualizing and evaluating surgery probe positions and directions. Even further, methods of the present inventions would provide fused images of data captured by any device attached to the head with internal brain anatomy and additionally with a brain functional activation map.

The inventors contemplate that fusion of data is extendable to repeat data and to date gathered by other sensors, providing the data information has a known geometric relationship to the face. Such that, in one embodiment, two individual face surface data sets, such as a face map, are fused together. In another embodiment, data comprising at least two common geometric points are fused with MRI captured data.

Further contemplated embodiments of the present invention include but are not limited to direct visualization of the geometric relations of the head surface, face surface and any devices attached to the head to the internal brain anatomy and/or brain functional activation maps; registration of magnetic resonance imaging (MRI) data and data derived from MRI to other sensors and the derived data, provided the information comprises a known geometric relationship to the face that is captured by a camera or imaging device, further comprising methods providing for stereotactic (or similar) neurosurgery planning methods, for example, a neurosurgeon would observe by visualization exactly where and in which direction to place a surgery probe, such that images would be static or real time, methods for registering and comparing functional brain activation maps captured from a functional MRI (fMRI) scan combined with event-related potential (ERP) data captured from an electroencephalogram (EEG) device further providing benefits of combining high-temporal resolution of EPR data and the high-spatial resolution of fMRI data. For example, the inventors are currently contemplating applying these methods in order to combine event-related potential (ERP) data with fMRI (as shown herein) [Mangun et al., Hum Brain Mapp, vol. 6, pp. 383-389, 1998; herein incorporated by reference].

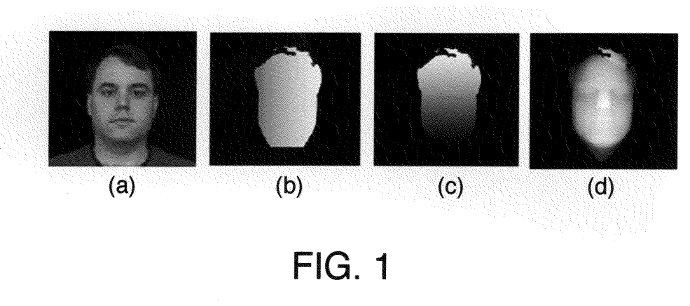

In summary, a direct visual feedback system contemplated herein is based on a system for fusing color 2.5D images of the human face with 3D MR brain images (FIG. 1). This system was recently developed by the inventors (Colbry et al., IEEE Transactions on Medical Imaging, 2007; herein incorporated by reference). This system is fast, robust, fully automated and highly accurate, and can be used to establish geometric relationships between brain anatomy and features of the face or head. The face images were generated with a digital laser scanner. The fusion process for combining the face scan and the 3D MR images took less than two seconds (using a computer with Windows XP, and a 3.2 GHz Pentium 4 processor). Alignment errors are very small, within 1.4±0.4 mm.

A real-time direct visual feedback system contemplated herein is outlined in a schematic in FIG. 8. Before this system is applied, high-resolution 3D MR brain images need to be captured (acquired) while artificial face surface maps and optional fMRI activation maps also need to be created. (See the Methods section, below, for details on the data acquisition, analysis and data fusion procedure).

During a neurosurgical planning procedure the surgeon marks the incision site using ink and/or by attaching a mechanical device to the patient's head. A digital laser scanner would then be used to acquire new 2.5D images of the patient's head, showing the incision markings and/or the device. Then the 2.5D images would be fused with the 3D MR images and the associated fMRI brain activation maps. The newly fused data set would then be visualized in three dimensions, providing direct visual feedback to the surgeon about whether the incision site and direction were marked correctly or if they need to be adjusted. In the next step, a simulated surgical path from the incision site to the target surgical region is projected, showing the direct connection between the two regions. This surgical path would be visualized along with the fused image dataset, providing real-time feedback (in seconds) for each incision site and direction that the surgeons seek to evaluate. Note that although a straight line projection may not be the actual cut in clinical practice, it should provide helpful feedback.

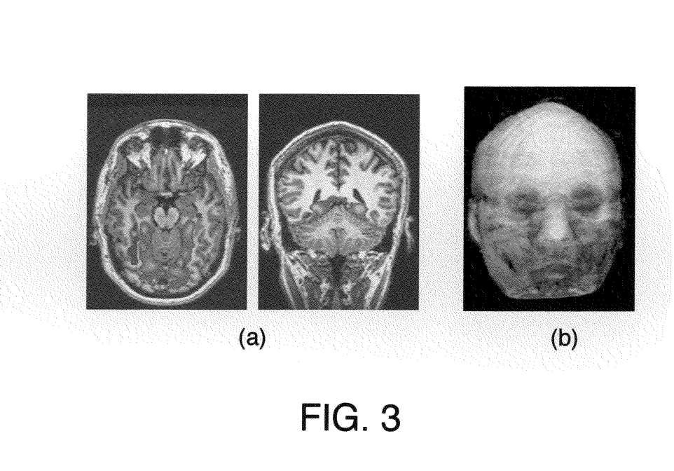

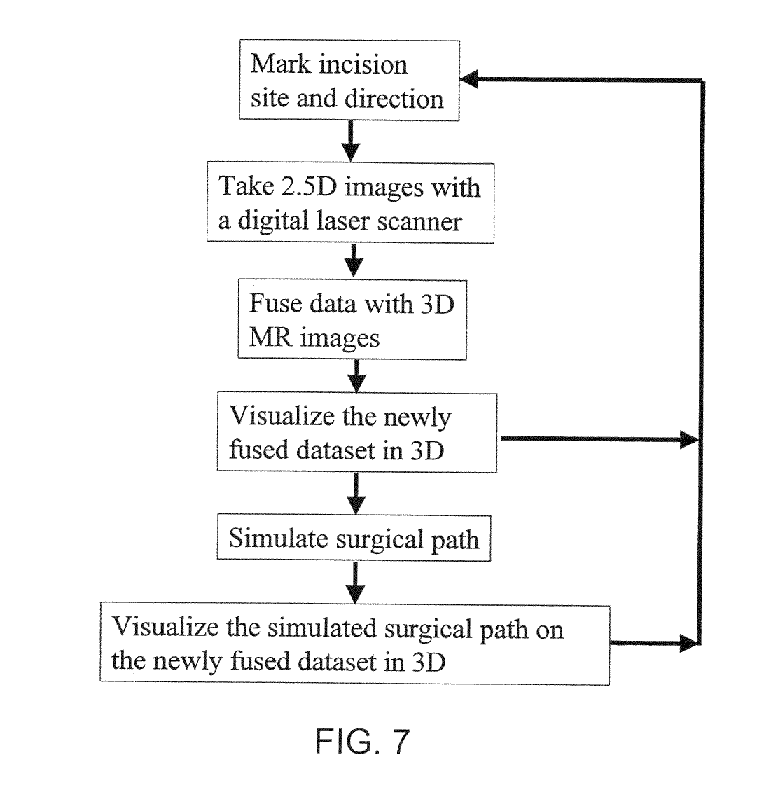

The most important component of the contemplated visual feedback system (see, for example, FIG. 8) is the data fusion process shown in FIG. 3. The core of the contemplated methods (shown inside the large rectangle) has been successfully prototyped (FIG. 7). During the experiment shown in FIG. 1, the addition of an ERP electrode cap did not cause difficulty during the data fusion, suggesting that an external mechanic device worn by a patient should not impede the data fusion process contemplated for these methods.

### Experimental

The following examples are provided in order to demonstrate and further illustrate certain preferred embodiments and aspects of the present invention and are not to be construed as limiting the scope thereof.

In the experimental disclosures which follow, the following abbreviations apply: cm (centimeters); mm (millimeters); μm (micrometers); nm (nanometers); U (units); min (minute); s and sec (second); ° and deg (degree); D (dimensional), optical density (OD), and volts (V).

### EXAMPLE I

### Materials and Methods

The following reagents and methods were used in the EXAMPLES described herein.

Subjects: A healthy 33-year old male volunteered to participate in this study. He signed Michigan State University Institutional Review Board approved consent forms.

### 2.5D Image Acquisition and Face Alignment.

2.5D images of the face were acquired with the Minolta “VIVID 910 Non-Contact 3D Laser Scanner,” using a “structure from lighting” method [Minolta, described at world wide web: konicaminolta.com/products/instruments/vivid/vivid910.html, 2005; herein incorporated by reference] that combined a horizontal plane of laser light with a 320×240 pixel color camera. As the laser moved across the surface being scanned, the color camera observed the curve produced by the interaction of the laser and the object. The scanner used this data to triangulate the depth of the illuminated surface points (with an accuracy of ±0.10 mm in fine resolution mode), resulting in a 2.5D scan (FIG. 1) that included both Cartesian (x, y, z) coordinates and color (red, green, blue) information for every pixel within the scan.

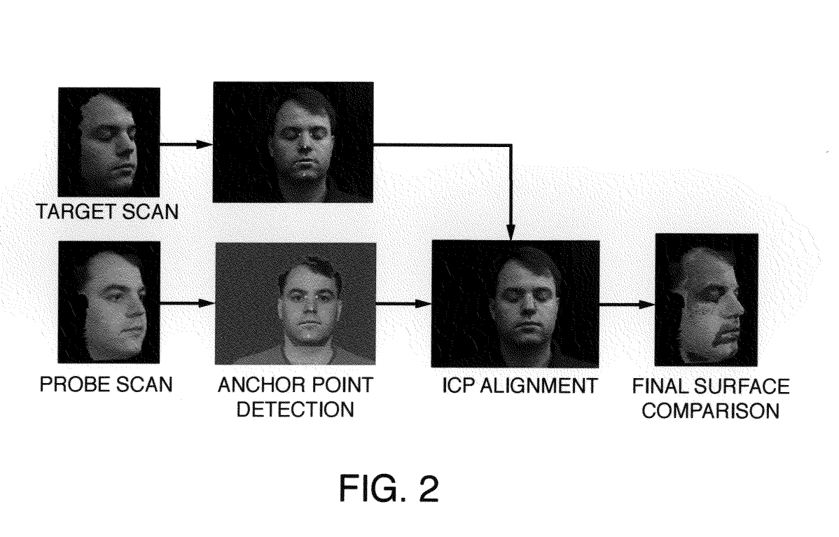

A 3DID face identification system was implemented in C++ on a Window XP platform (which was compatible with the Vivid 910 laser scanner). The 3DID software applies an automatic, two-step surface alignment algorithm (outlined in FIG. 2) to determine whether two 2.5D scans are from the same person. The first step is to coarsely align the two scans using automatically detected anchor points, such as the tip of the nose and the inside corners of the eyes. The anchor points are found automatically by comparing a generic model of the face with a pose invariant surface curvature [Colbry et al., “Detection of Anchor Points for 3D Face Verification,” in IEEE Workshop on Advanced 3D Imaging for Safety and Security A3DISS. San Diego Calif., 2005; herein incorporated by reference]. The second step finely aligns the two face scans using a hill-climbing algorithm called Trimmed Iterative Closest Point [Chetverikov et al., “The Trimmed Iterative Closest Point Algorithm,” in Proceedings of the 16th International Conference on Pattern Recognition, vol. 3. Quebec City, Canada, pp. 545-548, 2002; herein incorporated by reference] (tICP). The tICP algorithm takes a small set of control points (100 points in a grid around the nose and eyes) on one scan (the “probe” scan) and finds their nearest neighbors on the coarsely aligned surface of the second scan (the “target” scan). Next, 10% of the control points are trimmed to account for noise in the data (the trimmed points have the largest distance between the point and the surface of the scan). Finally, a 3D transformation is calculated to reduce the root mean squared distance error between the remaining 90% of the control points on the probe scan with the surface of the target scan. The alignment process repeated until the distance error falls below a minimum threshold or until the iteration limit (typically 10) was reached.

Using this technique, two face scans from the same person can be aligned in less than two seconds (Windows XP, 3.2 GHz Pentium 4 processor). For frontal scans with neutral expression, the alignment algorithm is immune to spike noise, missing surface points and lighting variations, and can tolerate pose variations of up to 15 degrees of roll and pitch, and up to 30 degrees of yaw [Stockman et al., Sensor Review Journal, vol. 26, pp. 116-121, 2006; herein incorporated by reference].

MRI and fMRI Data Acquisition and Analysis.

MRI and fMRI data were first acquired on a 3T GE Sigma EXCITE scanner (GE Healthcare, Milwaukee, Wis.) with an 8-channel head coil. This subject is from a 15-subject scene processing study [Henderson et al., Full scenes produce more activation than close-up scenes and scene-diagnostic objects in parahippocampal and retrosplenial cortex: An fMRI study. Brain and Cognition Brain and Cognition, 66, 40-49, 2008; herein incorporated by reference], where the data acquisition and analysis processes have been fully described. To study brain function, echo planar images were acquired with the following parameters: 34 contiguous 3-mm axial slices in an interleaved order, TE=25 ms, TR=2500 ms, flip angle=80°, FOV=22 cm, matrix size=64×64 and ramp sampling. During the brain function study, the subject was shown 120 unique pictures for each of the four conditions (Full Scenes, Close-up Scenes, Diagnostic Objects and Faces). The experiment was divided into four functional runs each lasting 8 minutes and 15 seconds. In each run, subjects were presented with 12 blocks of visual stimulation after an initial 15 s “resting” period. In each block, 10 unique pictures from one condition were presented. Within a block, each picture was presented for 2.5 s with no inter-stimulus interval. A 15 s baseline condition (a white screen with a black cross at the center) followed each block. Each condition was shown in three blocks per run. After functional data acquisition, high-resolution volumetric T₁-weighted spoiled gradient-recalled (SPGR) images with cerebrospinal fluid suppressed were obtained to cover the whole brain with 124 1.5-mm sagittal slices, 8° flip angle and 24 cm FOV. These images were used to identify detailed anatomical locations for functional statistical activation maps generated. They can be reconstructed to provide the face surface map of the skull, which was the data used to combine the MRI and fMRI data with the 2.5D face scans obtained by the laser scanner.

Functional fMRI data pre-processing and analysis were conducted with AFNI software Cox et al., Computers and Biomedical Research, vol. 29, pp. 162-173, 1996; herein incorporated by reference. The reference function throughout all functional runs for each picture category was generated based on the convolution of the stimulus input and a gamma function [Stockman et al., Sensor Review Journal, vol. 26, pp. 116-121, 2006; herein incorporated by reference], which was modeled as the impulse response when each picture was presented. The functional image data acquired were compared with the reference functions using the 3dDeconvolve software for multiple linear regression analysis and general linear tests [Ward et al., “Deconvolution analysis of fMRI time series data” Biophysics Research Institute. Milwaukee, Wis.: Medical College of Wisconsin, 2002; herein incorporated by reference]. The contrast based on the general linear test of Full Scenes over Faces was used as the statistical activation maps (voxel-wise p value<10⁻⁴ and a full-brain corrected p value<1.5×10⁻³) in this paper to demonstrate the application of the face-alignment technique.

Before the application of the face-alignment technique, both the high-resolution volumetric MR images and t-statistic brain activation maps were linearly interpolated to a volume of 240 mm×240 mm×180 mm with a voxel size of 1 mm×1 mm'1 mm (FIG. 3) with the AFNI software [Cox et al., Computers and Biomedical Research, vol. 29, pp. 162-173, 1996; herein incorporated by reference]. These high-resolution volumetric MR images were used in the face map fusion process, and the activation maps were overlaid.

### Fusing 2.5D Face Surface Scans and 3D MR Images.

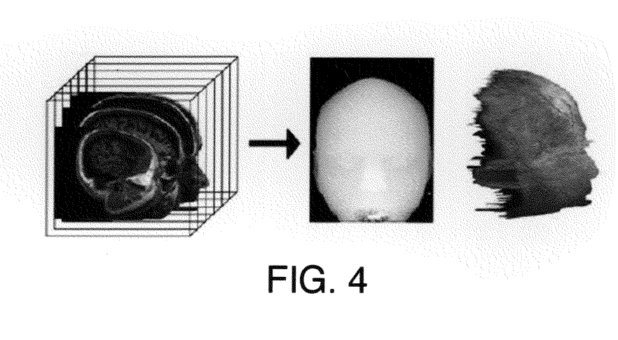

In one embodiment of the present invention, a 2.5D laser scan was fused with 3D MR images using the following method: an artificial 2.5D face surface scan was created from 3D MR images using the process illustrated in FIG. 4. First, voxel signal values were picked from the “face” region of the skull in the MR image (an estimated “skin” threshold value was determined manually based on the voxel signal intensity histogram which shows a clear separation between “skin” tissue and air regions). At each sagittal slice and at each row of a slice, the comparison started from the most anterior voxel to the most posterior voxel. Once the voxel signal intensity was above the threshold value, the distance from the most anterior voxel of the slice was recorded. Repeating this procedure for all slices and all rows in each slice, a 2.5D surface image of the face was created based on the distance to the anterior edge of the brain image volume. Finally, the alignment algorithm from 3DID was used to align the artificial 2.5D face surface scan from the 3D MR images to the actual 2.5D face surface scan generated by the laser scanner.

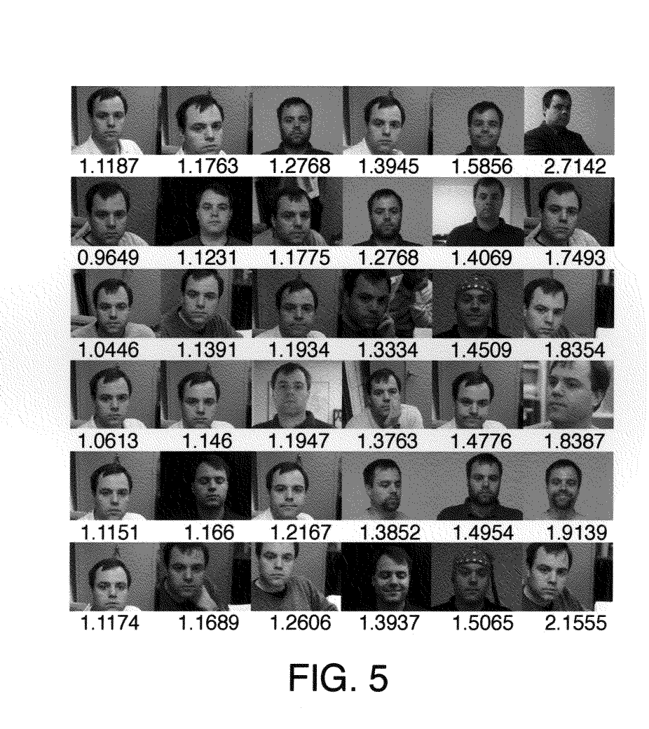

In another embodiment of the present invention, a method to evaluate the robust nature of the fusion technique to MRI was used where this method was applied to 36 laser 2.5D scans of the test subject. These 2.5D scans were taken over a period of four years, varying widely in pose and expression, and even including changes in facial hair (FIG. 5).

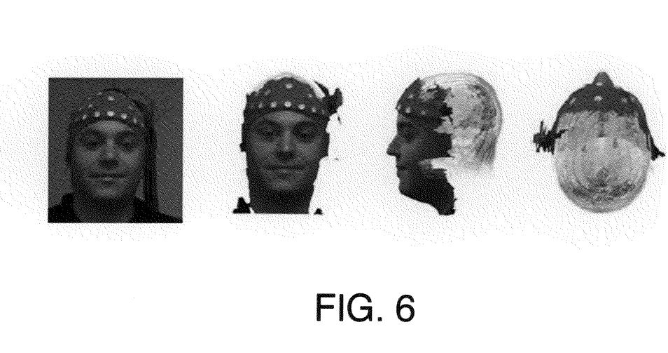

Additional valuable information can be included in the fused data. For example, (FIG. 6) shows the results of fusing MR images with a brain statistical activation map overlaid and laser scans when the subject is wearing an ERP electrode cap.

### EXAMPLE II

A face alignment algorithm developed for 3DID was extended to combine 2.5D face scans from a laser scanner with 3D MR images. Based on the evaluation of the 36 laser 2.5D scans of the test subject (FIG. 6), the alignment error is 1.4±0.4 mm and takes less than 2 seconds to complete the alignment process for each scan on a personal computer (Windows XP, 3.2 GHz Pentium 4 processor), which can be obtained easily in the current market and costs about $1000).

This alignment technique could be expanded to allow the combination of any data that has a known relationship to the surface of the human face. For example, features of the face and head (and any attached devices or frames) could be directly visualized in relationship to the brain anatomy and/or brain activation maps. This technique would be valuable for stereotactic (or similar) neurosurgery planning methods [Hunsche et al., Phys Med Biol, vol. 42, pp. 2705-2716, 2004; herein incorporated by reference]. The inventor's ongoing work includes fusing data obtained from ERP experiments with fMRIs, allowing the inventor's to combine high-temporal resolution of EPR data with the high-spatial resolution of fMRI data.

All publications and patents mentioned in the above specification are herein incorporated by reference in their entirety. Various modifications and variations of the described method and system of the invention will be apparent to those skilled in the art without departing from the scope and spirit of the invention. Although the invention was described in connection with specific preferred embodiments, it should be understood that the invention as claimed should not be unduly limited to such specific embodiments. Indeed, various modifications of the described modes for carrying out the invention that are obvious to those skilled in biometry, physics, neurosurgery, chemistry, molecular biology, medicine or related fields are intended to be within the scope of the following claims.

We claim:

1. A system, comprising software, wherein said software is configured to:
i) receive a first data set obtained from a digital laser scanner;
ii) receive a second data set obtained from a magnetic resonance imaging (MRI) device; and
iii) fuse said first data set and said second data set into a fused data set.

2. The system of claim 1, wherein said software is further configured to display said fused data set.

3. The system of claim 2, wherein said first data set comprises a human head and neck scan.

4. The system of claim 3, wherein said first data set further comprises a human face scan.

5. The system of claim 4, wherein said second data set comprises at least one brain anatomy image.

6. The system of claim 4, wherein said second data set comprises at least one brain activation image.

7. The system of claim 5, wherein said fused data comprises said face and head scan superimposed with said brain anatomy image.

8. The system of claim 6, wherein said fused data comprises said a face and head scan superimposed with a brain activation image.

9. The system of claim 1, wherein said digital laser scanner device is a facial scanner device.

10. The system of claim 1, wherein said second data set obtained from a magnetic resonance imaging (MRI) device is in real-time.

11. The system of claim 1, wherein said first data set and said second data set sample are obtained from the same subject.

12. The system of claim 5, wherein said anatomy image comprises an abnormal cell image.

11. The system of claim 6, wherein said activation image comprises an abnormal activation image.

13. A system, comprising:
a) a digital laser scanner device,
b) a magnetic resonance imaging (MRI) device,
c) a first data set and a second data set, wherein said first data set comprises sample data obtained by a digital laser scanner device and said second data set comprises sample data obtained by a magnetic resonance imaging (MRI) device,
d) software, wherein said software is configured to:
i) receive a first data set obtained from a digital laser scanner;
ii) receive a second data set obtained from a magnetic resonance imaging (MRI) device; and
iii) fuse said first data set and said second data set into a fused data set.

14. The system of claim 13 wherein said software is further configured to display said fused data set.

15. A method of generating fused sample data, comprising,
a) providing,
i) a first data set obtained from a digital laser scanner device,
ii) a second data set obtained from a magnetic resonance imaging (MRI) device, and
b) combining said first sample data set and said second sample data set so as to generate a fused sample data set.

16. The fused sample data set generated according to claim 15.

17. A method of fusing sample data, comprising,
a) providing,
i) a first data set obtained from a digital laser scanner device,
ii) a second data set obtained from a magnetic resonance imaging (MRI) device, and
iii) a software package configured to fuse sample data sets, and
b) combining said first sample data set and said second sample data set using said software package for providing a fused sample data set.

18. The method of claim 17, further comprising, iv) a software package capable of displaying a fused data set and c) displaying said fused sample data set.

19. The method of claim 17, wherein said first data set comprises a human head and neck scan.

20. The method of claim 19, wherein said first data set further comprises a human face scan.

21. The method of claim 20, wherein said second data set comprises at least one brain anatomy image.

22. The method of claim 21, wherein said second data set comprises at least one brain activation image.

23. The method of claim 22, wherein said anatomy image comprises an abnormal cell image.

24. The method of claim 23, wherein said activation image comprises an abnormal activation image.

25. The method of claim 24, wherein said fused data comprises said face and head scan superimposed with said brain anatomy image.

26. The method of claim 25, wherein said fused data comprises said face and head scan superimposed with said brain activation image.

27. The method of claim 17, wherein said digital laser scanner device is a facial scanner device.

28. The method of claim 17, wherein said sample data set obtained from a magnetic resonance imaging (MRI) device is real-time data.

29. The method of claim 17, wherein said first data set and said second data set sample are obtained from the same subject.

30. The method of claim 17, further comprising, a third sample data set obtained from an event-related potential (ERP) electrode cap and combining said third sample data set with said software for providing a fused sample data set.

31. A method, comprising,
a) providing,
i) a human patient, wherein said human patient is in need of a neurosurgical procedure,
ii) a surgical incision marker, wherein said marker is capable of showing the location of a planned incision site and the surgical direction of a planned incision site, wherein said marker is capable of being digitally scanned on a human patient,
iii) a 3D data set, wherein said 3D data set was obtained by scanning a human patient,
iv) a digital laser scanner, capable of generating a 2.5D data set from a human patient, and
v) a software package configured to fuse and display a 2.5D data set fused with a 3D data set, wherein said software is further configured to simulate a surgical path on said display,
b) marking the location of a planned incision site and the surgical direction of incision with said marker on said human subject,
c) scanning said marked patient with said scanner for obtaining a 2.5D data set,
d) fusing said 2.5D data set with said 3D data set with said software package,
e) displaying said fused data sets, and
f) simulating a surgical path on said display for a neurosurgical procedure.

32. The method of claim 31, wherein said 3D data set was obtained by a magnetic resonance imaging (MRI) device for capturing said 3D data set from said human subject.

33. The method of claim 31, further comprising, erasing said marking and repeating steps b)-f), wherein said planned incision site is changed.
